(19) United States
(12) Patent Application Publication
(10) Pub. No.: US 2025/0201376 A1
(43) Pub. Date: Jun. 19, 2025

(54) Reusable Digital Metered Syringe

(71) Applicants: Nemalin Tharushen Perumal, Singapore (SG); Jaishri Ellengovan Desikar, Singapore (SG)

(72) Inventors: Nemalin Tharushen Perumal, Singapore (SG); Jaishri Ellengovan Desikar, Singapore (SG)

(73) Assignee: BOXTHREE PTE. LTD, Singapore, SG (SG)

(21) Appl. No.: 18/539,289

(22) Filed: Dec. 14, 2023

(51) Int. Cl. G16H 20/13; A61M 5/20; A61M 5/31; A61M 5/32

(52) U.S. Cl. CPC G16H 20/13; A61M 5/2053; A61M 5/3202; A61M 2005/3126

(57) ABSTRACT

In some embodiments thereof, the present invention discloses a reusable smart syringe for radically enhanced liquid medication delivery through integrated digital measurement, dosage guidance and tracking technology. In some aspects thereof, an optical scanner reads embedded prescription codes on medicine labels to automatically recognize and set required dispensed volumes. Precision fluid amount sensing and electronic control circuitry coupled to a digital display panel enables exact fluid volume measurement and user verification improving upon imprecise analog techniques. Dosage volumes are also digitally limited as per scanned prescription codes to prevent under/overfills. An ergonomic thumb-press facilitates smooth one-handed operation alongside recyclable, detachable nozzle tips reducing medical plastic waste. All dispensed events are logged electronically interfacing with patient records for intelligent medication compliance improvement. The inventive integration of digital enhancements including computer vision, electronics, data connectivity and ergonomic engineering within legacy syringes establishes the next generation of safe, effective smart medication delivery advancing patient outcomes.

### FIELD OF INVENTION

The present invention relates to smart medication delivery systems, specifically improved electronic fluid dispensers incorporating sensing, digitization, dosage control and connectivity. The invention discloses novel syringes with integrated sensors, motors, displays and data capabilities enhancing legacy devices for radically advanced accuracy, usability, waste reduction and patient compliance monitoring to provide the next level in safe, effective smart liquid medication dosage.

### BACKGROUND OF THE INVENTION

Liquid medication dosage delivery devices such as syringes and droppers have existed for over a century, providing a simple mechanism to draw and dispense precise fluid medication volumes. While many advances have improved drug efficacy, the core fluid dispensing methods remain relatively unchanged until the arrival of smart ‘connected’ medical devices. As such, existing dispensing techniques and technology have critical limitations that impact accuracy, ease-of-use, waste and compliance-ultimately reducing treatment effectiveness.

While the medical syringe market alone is estimated to generate $9.24 billion by 2028, the existing syringe technology landscape provides insufficient accuracy and precision in liquid measurement capabilities for optimal treatment. Both the more primitive glass and disposable plastic syringes rely on imprecise manual measurement via visual alignment with printed scaling. This ad-hoc technique introduces significant margin of human error and inability to control dispensed amounts, with volume readings often inaccurate by over 10%. Such imprecision leads directly to the critical medical challenge of accidental under and overdosing, causing treatment ineffectiveness or patient harm.

Furthermore, existing syringes require good manual dexterity and strength for smooth plunging motion to suction and dispense liquids. This poses severe difficulties in operation by children, elderly and disabled users with limited strength or motor control. The associated lack of ergonomic considerations reduces system usability and accessibility for a significant demographic of end users.

Additionally, with no integrated dosage guidance, users must manually refer to prescription guidelines to set the appropriate medication amounts. This is cognitively burdensome and error-prone, again increasing chances of incorrect dosage. There also exists no digitized data capture of dispensed amounts, preventing detailed tracking of patient medicine intake for compliance monitoring and improvement.

While ‘smart’ syringes using modern sensors, motors and electronics exist in academic studies, these have yet to reach commercial viability and mass adoption. As such there remains a strong unmet market need to enhance the legacy syringe with integrated digital enhancements to transform accuracy, user experience, waste and data connectivity. Tremendous opportunity exists to reinvent this age-old device via sensors, displays, connectivity and dosage helpers. By intelligently bridging the analog and digital worlds the smart syringe can provide the next leap in safe, effective medication delivery.

### SUMMARY OF THE INVENTION

The following summary is an explanation of some of the general inventive steps for the invention in the description. This summary is not an extensive overview of the invention and does not intend to limit its scope beyond what is described and claimed as a summary.

In some aspects thereof, the present invention discloses a smart digitally-integrated syringe system to transform legacy devices for significantly enhanced medication fluid dispensation accuracy, ergonomics, dosage control and monitoring. A novel reusable syringe is introduced comprising integrated sensors, motors, display and data exchange capabilities.

In another aspect, a scanner reads unique medicine prescription code labels, identifying the precise required dosage for automated syringe volumetric control. The scanner communicates detected dosage data to the syringe's control circuitry. This innovative optical recognition and control system ensures accurate, personalized medicine quantities are dispensed adapted per patient needs, significantly improving dosing precision over imprecise manual techniques prone to human error.

Further improving ease-of-use, an ergonomic thumb-press enables smooth one-handed operation for children or motion-impaired users. This alleviates manual force and dexterity challenges existing devices pose. The syringe nozzle also detaches for simplified washing, fitted with recyclable biopolymer tips reducing medical plastic waste.

Additionally featured is a digital fluid measurement system constituting vacuum chamber fluid displacement sensors and electronic control circuitry. It precisely gauges suctioned liquid volumes, displaying the value on a backlit panel for user verification. This enhances volume visibility and calibration, augmenting basic manual physical scale readings.

In a non-limiting embodiment, a dosage control system also intelligently limits fluid draw volumes per the scanned prescription code data for accurate, personalized dispensation. This prevents potentially dangerous ad-hoc estimates or overfill. Further, all dispensed amounts are digitally logged on device memory interfacing with patient medical records. This enables intelligent medication compliance tracking and pattern analysis for usage monitoring and improvement.

In a non-limiting aspect, the integrated technological enhancements transform primitive analog syringes into smart, precise, ergonomic liquid medication delivery systems. The inventive scanner, sensors, display and connectivity establish the next generation of safe, effective medical fluid dispensers advancing patient wellbeing.

### BRIEF DESCRIPTION OF THE DRAWINGS

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 depicts a conceptual structure of a reusable digital metered syringe in accordance with an aspect of this disclosure.

FIG. 2 demonstrates a digital scanner using the integrated optical scanner to read unique prescription codes that identify required dosage volumes in accordance with an aspect of this disclosure.

FIGS. 3A and 3B illustrates various components of a reusable digital metered syringe in accordance with an aspect of this disclosure.

FIG. 4 shows a process flow diagram outlining the software functionality and digital operations enabling smart precise fluid measurement, prescription code scanning, dosage control, volumetric dispensing and logged data history.

### DETAILED DESCRIPTION OF THE PREFERRED EMBODIMENTS

Hereinafter, the preferred embodiment of the present invention will be described in detail and reference made to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

It is to be understood that the form of the invention shown and described herein is to be taken as a preferred embodiment of the present invention, so it does not express the technical spirit and scope of this invention. Accordingly, it should be understood that various changes and modifications may be made to the invention without departing from the spirit and scope thereof.

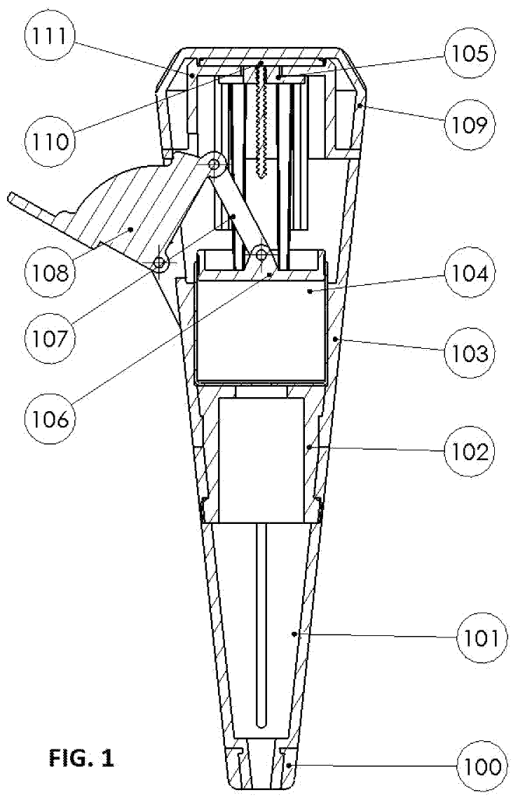

The non-limiting embodiment according to FIG. 1 illustrates a conceptual reusable digital syringe comprising multiple key components enabling enhanced fluid medication dosage and intelligent monitoring. A disposable nozzle tip (100) attaches to the nozzle (101), temporarily storing suctioned liquids. In a preferred embodiment, contemporary rigid plastic tips posing environmental waste concerns are replaced with novel biodegradable cornstarch polymers or recycled paper pulp materials. Integrating green chemistry principles reduces medical plastic pollution without performance trade-offs.

The nozzle houses an electronically-controlled non-return valve system (102). In a preferred embodiment, contemporary mechanical valves demonstrating sticking from medication residue buildup are upgraded with premium electroactive polymer actuators for reliable sensitive response. Controlled by device circuitry, this intelligently governs fluid inlet/outflow.

Preferably, the components are encased in a watertight ergonomic enclosure (103), maximizing grip comfort. In some preferable aspects, human factor studies determine optimal grip shapes suiting diverse hand anthropometrics. In a preferred aspect, the enclosure dynamically senses handling, heating/cooling for comfort.

Within is a vacuum chamber (104) partially evacuated to enable fluid suction powered by an electric motor compressed spring assembly. The spring syringe subclass is selected over manual variants for its superior ergonomics from electronically-assisted operational force reduction. It may be preferred to enable multiple, separate doses to be pre-filled into detachable chambers in advance for later dispensing convenience. As such, according to one non-limiting aspect, the chamber may comprise a suitably-shaped container that screw-interlocks with the nozzle assembly via an integrated threaded male connection mating a female threaded receptor. Such a threaded junction may integrate raised chevroned gaskets on both components, enabling a tight irrotational seal when fully connected.

As an example, an empty chamber module twists into the nozzle loading position and the plunger automatically draws in a calibrated dose based on the scanned prescription. When filled, the chamber rigidly detaches by unscrewing while remaining fluid-enclosed via an integrated non-return valve impeder. The non-return valve may comprise a passive spring-loaded conical stopper only permitting inward fluid flow. Multiple filled chambers be accumulated ready for use. Subsequently when dispensation is required, a selected prefilled chamber module simply twists back onto the nozzle receptacle, the non-return valve easily opening upon reattachment to enable bidirectional flow. The extendable dispensing button fully depresses the chamber internal plunger evacuating contents through the nozzle under precise volumetric control.

The limiting link (105) sets the permissible range of motion of the plunger (106), regulating the maximum travel distance and dispensed fluid amount. It mechanically interconnects the plunger, press and controller cap components while allowing detachment for service. Depending on syringe calibration flexibility requirements, the limiting link can comprise fixed rigid bodies to strictly govern motion, or incorporate tool-adjusted screw assemblies to enable adjustable dosing control.

The plunger (106) displaces a precise fluid volume dictated by the controlled linear actuation. Integrated sensing elements within the plunger measure its displacement and the corresponding volume of liquid transferred, feeding sensor data to the control circuit for closed-loop feedback dosage metering. While typically plastic-based as in legacy art, the plunger can alternatively employ ceramic, metallic or more exotic materials that meet dimensional stability, chemical resilience and manufacturability constraints.

The press (108) and plunger (106) interface via a low-friction connector (107) allowing sliding action for smooth linear actuation yet permitting detachment for maintenance. The connector employs slippery materials such as Teflon at the interfacing surfaces to mitigate wear from repeated actuator strokes. Internally, the press houses motors and control hardware to govern plunger motion, enabling precise electronically-controlled fluid amounts. Embedded force sensors provide feedback mitigating excessive insertion pressures, preventing patient harm.

The controller cap (109) adjusts the plunger travel range through height calibration, increasing or lowering dispensed volumes. Manual finger manipulation or assisted tool-based actuation options exist for precision setting. The integrated limit screw (110) transfers these adjusted displacement thresholds to the limiting link (105). A detachable coupler (111) allows convenient controller cap rotation then rigidly fixes elements post-calibration to resist displacement during syringe operation.

Fluid volume measurement and calibrated dosing is enabled by an integrated sensor-assisted plunger (106) and link mechanism (105). Optical, ultrasound or laser time-of-flight fluid level sensors feed data to control circuitry, enabling precise volumetric metering. Stepper motors allow electronically configurable travel distance to meet prescribed amounts.

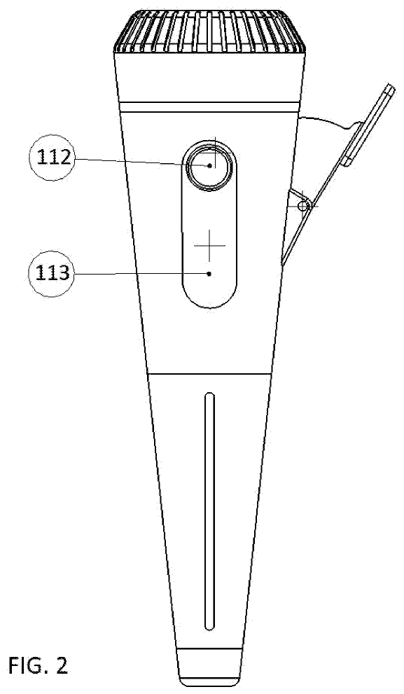

In another aspect, FIG. 2 demonstrates digital scanner using the integrated optical scanner to read unique prescription codes that identify required dosage volumes in accordance with an aspect of this disclosure. The inclusion of an integrated optical scanner (112) and control circuit (113) enable automated recognition of medication fluids and accurate dosage control. Preferably, the digital scanner comprises an array of visible or infrared optical sensors coupled to illumination elements scanning across imprinted labels on medicine bottles and packages during the dispensing process. Sensor resolutions (preferably of 200 dpi or greater) resolve small text/codes including industry standard barcode formats like UPC and QR codes or proprietary medication data matrices readable at close distances. Imaging hardware costs continue rapid decline enabling integration.

In some aspects, embedded barcode data particularly provides unique fluid identifiers, while code database look-ups enable extraction of medication instructions and parameters from remote digital datasets in vendor EHR systems. These instruct syringe control circuitry of required dosage amounts for a given patient fill prescription. Barcode uptake across pharmaceuticals presently exceeds 80% enabling broad applicability.

Control circuits manipulate embedded micro-controllers, data converters, memory and I/O hardware resources to orchestrate device behaviors based on scanned data inputs. Micro-controllers govern core functions from fluid sensing, pumping, valves and user interfaces while maintaining safe operating thresholds. Circuits integrate valve/pump actuation and measurements from displacement and pressure sensors to enforce proper fill levels. User interface provides confirmation and any manual dosage adjustment if required.

Positively, the embodiment of FIG. 2 providing the scanner coupled to intelligent circuitry automation and integration with remote prescribing medical records provides a major leap toward safe pharmacy-grade precision and dosage control compared to fully manual techniques prone to massive human error. Users now simply scan a label rather than guesswork ad-hoc adjustments.

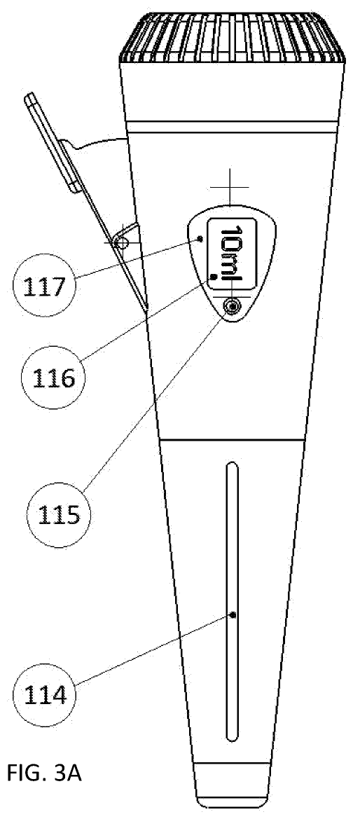

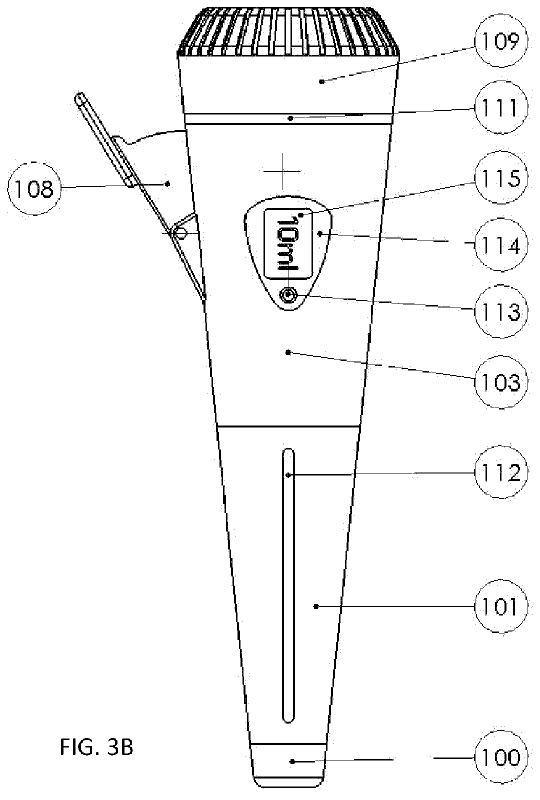

Further, the non-limiting embodiment of FIGS. 3A and 3B illustrate additional syringe components improving ergonomics, visualization and system control. Firstly, FIG. 3A exhibits the fluid gauge (114) which utilizes physical indicators to supplement the digital volumetric measurement, providing secondary verification of drawn amounts through direct visual inspection by a user. Analog indicators remain useful in the case of electronic sensor discrepancies. The gauge can comprise transparent chambers with physical level lines, floating component trackers, or tubes with pellet stacks that reduce in height based on fluid offsets.

Also shown is the power button (115) allowing manual activation and deactivation of device functionality alongside sleep/wake modes for energy efficiency. The integrated digital panel (116) and display circuit (117) work in conjunction to show measured fluid amounts on graphical user interfaces intelligible at a glance. Sizeable LED or LCD screens with high contrast ratios allow legibility for those with visual impairments. Touch capabilities are also increasingly integrated for enhanced convenience and utility.

Further, FIG. 3B demonstrates the nozzle and tip system highlighting the key smart syringe differentiators. The detachable closing tip (100) attaches securely to the nozzle (101) for storage and safety, manufactured using sustainable biopolymers which can be recycled and reused reducing medical plastic waste.

The ergonomic press (108) has been purposefully contoured and sized for convenient single-handed thumb operation by users with limited strength and motor control like children. No manual squeezing or high force required compared to incumbent devices. The control cap (109) employs a quick turn coupler (111) for tool-less volume limit adjustment then rigid locking at desired calibration. Overall the embodiment discloses both assistive and sustainability oriented advances easing adoption across wider demographic groups.

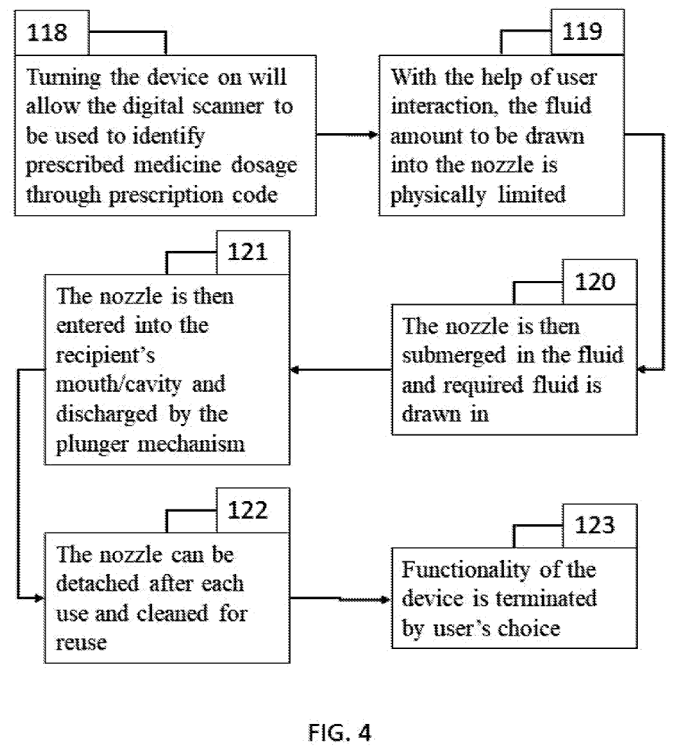

The last shown embodiment according to FIG. 4 demonstrates a a process flow diagram outlining the reinvented digital functionality and software-based operation of the inventive smart syringe system constituting a significant advancement over conventional manual approaches. The operational sequence initiates with the user powering on the device via a button press in a process step 118. This activates onboard controller hardware and sensing systems, mechanical pumps and values, user interfaces and optical prescription code scanning elements. Comprehensive initialization self-tests verify all components function within calibrated tolerances.

Subsequently, the user presents a medication bottle imprinted with encoded dosage data to the scanner window a process step 119. Optical sensors capture label imagery for machine vision processing to extract embedded product identifiers, drug names and dosage parameters into digital variables. Cloud database lookups further enrich medicine metadata as needed.

Further, extracted dosage data automatically configures permissible displacement limits imposed on the plunger pump assembly matched to precise amounts required a process step 120. This prevents potentially dangerous overfills beyond required quantities. The user then immerses the nozzle tip and presses the ergonomic button to trigger automated plunger retraction governed to the configured fill limit for safety. Integrated flow sensors measure intake for graphical display feedback.

Even further, internal mechanical switches toggle configuring the system from the intake phase into a calibrated dispensation phase. The user targets the recipient mouth region and hits the trigger initiating smooth automated plunger actuation calibrated not to over-dispense in a process step 121. Exact pre-configured amounts get reliably delivered.

In some aspects, all sensor measurements and operational events get logged locally a process step 122 for periodic synchronization to patient records, enabling fluid intake analytics. Nozzle sterilization reminder alarms also activate before the user powers down the device in a process step 123 into a dormant low power state, maximizing operation intervals between charges.

Embodiments of the smart syringe may utilize a variety of materials for constructing the external housing and internal components interfacing with the dispensed fluids when in operation. Materials are selected for mechanical properties enabling reliable precise function including dimensional stability, resilience to wear and chemical corrosion.

Alternative embodiments may selectively specify different materials for key sub-assemblies based on specialized performance requirements. The nozzle assembly in particular necessitates bio-compatibility for extensive direct fluid contact. The controller housing however demands durability and toughness to protect delicate interior electronics.

Additional embodiments also encompass smart syringes producible in a multiplicity of sizes, capacities and ergonomic shapes matched to usage scenarios from home healthcare to professional clinical settings. Configurability also suits variable hand sizes across potential consumer demographics including children and elderly with limited grip strengths.

Method alternatives extend to mechanical, adhesive or socketed fixturing of syringe components to ensure proper alignment, assembly and precision tolerance conformance during manufacturing procedures. Quality assurance processes further verify sensor calibration, control accuracy and fluid flow precision prior batch release.

The applicant intends to encompass all such obvious alternative designs, modifications and enhancements tailored for this smart syringe application that build on the core inventive concepts disclosed. References to singular terms are meant to additionally encompass plural forms, and vice versa, unless explicitly stated otherwise. Grammatical conjunctions denote both conjunctive and disjunctive combinations, unless otherwise evident from the context.

### INDUSTRIAL APPLICATION

The disclosed invention has broad applicability across the global healthcare industry for enhanced delivery and tracking of liquid medications. Its integrated precision fluid measurement, automated dosage control and sensor-basedlogging capabilities transform safety and transparency in medical fluid dispensation over primitive manual techniques. Industrial markets ranging from hospital pharmacies to home healthcare providers can incorporate these next-generation smart syringes improving dosing accuracy, user experience and medicine compliance monitoring-ultimately advancing patient outcomes through modern technology integration with incumbent devices. The invention also paves way for further smart medical instrument advancements interfacing biology, electronics and informatics.

What is claimed is:

1. A smart syringe for dispensing liquid medicines comprising:
a prescription code scanner configured to identify a medicine dosage by scanning a unique prescription code;
a control circuit configured to receive the identified medicine dosage from the prescription code scanner and control a dispensed volume of liquid medicine based on the received dosage; and
a fluid volume display configured to display the dispensed volume.

2. The smart syringe of claim 1, wherein the prescription code scanner optically scans and decodes the unique prescription code imprinted on a medicine label.

3. The smart syringe of claim 1 further comprising a nozzle for holding dispensed liquid medicine, the nozzle covered by a detachable tip.

4. The smart syringe of claim 3 further comprising a non-return valve for sealing the nozzle when detached and automatically released when the nozzle is attached for fluid dispensing.

5. The smart syringe of claim 1 further comprising a vacuum chamber giving way to a plunger, the plunger linked to an electrically controlled press for calibrated displacement of a fluid volume dictated by the control circuit.

6. The smart syringe of claim 5 further comprising a controller cap adjustable to set a maximum permitted displacement limit of the plunger, restricting a maximum dispensed fluid volume.

7. The smart syringe of claim 1 further comprising a physical fluid volume gauge allowing visual verification of digitally measured dispensed amounts.

8. The smart syringe of claim 1 further comprising a power button interfacing with the control circuit for manual powering on/off of the syringe.

9. The smart syringe of claim 1 further comprising a detachable chamber module configured to twist interlock with a mating threaded nozzle receptacle when filled with a calibrated fluid volume per identified dosage requirements and twist unlocked for detachment after filled.

10. The smart syringe of claim 9, wherein the detachable chamber module encloses an integrated non-return valve enabling unidirectional inward fluid intake as the chamber fills without leakage when subsequently detached.

11. A method for dispensing liquid medicines with a smart syringe, comprising:
scanning a unique prescription code on a medicine label using a prescription code scanner integrated into the smart syringe;
identifying a medicine dosage based on the scanned prescription code;
transmitting the identified medicine dosage to a control circuit within the smart syringe;
controlling a dispensed volume of liquid medicine by the control circuit based on the received dosage information; and
displaying the dispensed volume on a fluid volume display integrated into the smart syringe.

12. The method of claim 11, wherein the prescription code scanner performs optical scanning and decoding of the unique prescription code imprinted on a medicine label.

13. The method of claim 11, further comprising dispensing liquid medicine through a nozzle covered by a detachable tip.

14. The method of claim 11, further comprising automatically sealing the nozzle with a non-return valve when detached and releasing the valve when the nozzle is attached for fluid dispensing.

15. The method of claim 11, further comprising utilizing a vacuum chamber leading to a plunger within the smart syringe.

16. The method of claim 15, wherein the plunger is linked to an electrically controlled press for calibrated displacement of a fluid volume based on instructions from the control circuit.

17. The method of claim 15, further comprising adjusting a controller cap to set a maximum permitted displacement limit of the plunger, thereby restricting a maximum dispensed fluid volume.

18. The method of claim 11, further comprising digitally measuring the dispensed amount of liquid medicine using the smart syringe's control circuit.

19. The method of claim 11, further comprising interfacing a power button with the control circuit in the smart syringe.
